(19) United States
(12) Patent Application Publication
(10) Pub. No.: US 2011/0306516 A1
(43) Pub. Date: Dec. 15, 2011

(54) METHODS FOR PRODUCING INDUCED PLURIPOTENT STEM CELLS

(75) Inventors: David J. Kahler, New York, NY (US); Faizzan S. Ahmad, Fresh Meadows, NY (US)

(73) Assignee: THE NEW YORK STEM CELL FOUNDATION, New York, NY (US)

(21) Appl. No.: 13/159,030

(22) Filed: Jun. 13, 2011

Related U.S. Application Data
(60) Provisional application No. 61/354,987, filed on Jun. 15, 2010.

(51) Int. Cl. C40B 30/04; C12N 5/071; C12Q 1/68; C12Q 1/04; G01N 33/566

(52) U.S. Cl. 506/9; 435/34; 435/7.21; 435/6.12; 435/6.11; 435/325; 435/6.1

(57) ABSTRACT

The invention provides improved methods for producing induced pluripotent stem cells (iPSC) from adult fibroblasts. The methods include contacting adult fibroblasts with a reprogramming composition suitable for reprogramming the adult fibroblasts to iPSC, under conditions effective for the reprogramming composition to penetrate the adult fibroblasts, followed by culturing the contacted fibroblasts for a time period sufficient for the cells to be reprogrammed. The cultured cells are then sorted to select cells based upon their expression of the cell membrane surface markers CD13^{NEG} SSEA4^{POS} Tra-1-60^{POS}. iPSC colonies are then identified from the sorted cells.

This application claims the benefit of U.S. provisional patent application No. 61/354,987, filed on Jun. 15, 2010, incorporated herein by reference in its entirety.

### FIELD OF THE INVENTION

The invention is directed to improved methods for producing induced pluripotent stem cells (iPSC) from fibroblasts. More specifically the invention is directed methods for producing iPSC from adult skin fibroblasts by reprogramming adult fibroblasts and identifying the reprogrammed adult fibroblasts among other cells to identify those cells carrying certain markers.

### BACKGROUND OF THE INVENTION

Stem cells are =specialized cells that self-renew for long periods through cell division, and can be induced to differentiate into cells with specialized functions. These qualities give stem cells great promise for use in therapeutic applications to replace damaged cells and tissue in various medical conditions. Embryonic stem (ES) cells are derived from the blastocyst of an early stage embryo and have the potential to develop into endoderm, ectoderm, and mesoderm (the three germ layers) (i.e., they are “pluripotent”). In vitro, ES cells tend to spontaneously differentiate into various types of tissues, and the control of their direction of differentiation can be challenging. There are unresolved ethical concerns that are associated with the destruction of embryos in order to harvest human ES cells. These problems limit their availability for research and therapeutic applications.

Adult stem (AS) cells are found among differentiated tissues. Stem cells obtained from adult tissues typically have the potential to form a more limited spectrum of cells (i.e., “multipotent”), and typically only differentiate into the cell types of the tissues in which they are found, though recent reports have shown some plasticity in certain types of AS cells. They also generally have a limited proliferation potential.

Induced pluripotent stem cells (iPSC) are widely recognized as important tools for conducting medical research. Heretofore, the technology for producing iPSC has been time consuming and labor-intensive. Differentiated adult cells, e.g., fibroblasts, are reprogrammed, cultured, and allowed to form individual colonies which represent unique clones. Identifying these types of cells is difficult because the majority of the cells are not fully reprogrammed iPSC clones. The iPSC clones are then selected based on the morphology of the cells with “good” colonies possesing sharply demarcated borders containing cells with a high nuclear to cytoplasmic ratio. When clones are identified, they are manually picked by micro thin glass tools and cultured on “feeder” layers of cells typically Murine Embryonic Fibroblasts (MEF). This step is performed typically at 14-21 days post infection. Then the clones are expanded for another 14-21 days or more prior to undergoing molecular characterization.

Others have focused on developing techniques to rapidly and more accurately identify and characterize fully reprogrammed adult fibroblasts and their downstream differentiation potential (Bock et al., 2011, Cell 144: 439-452; Boulting et al., 2011, Nat Biotechnol 29: 279-286). These techniques include the use of Flow Cytometry (FC) and Fluorescence Activated Cell Sorting (FACS) to identify and live sort unique subpopulations of cells as defined by unique expression patterns of surface proteins.

Although FC and FACS are firmly established techniques in the field of immunology, their use in the stem cell field is still in its infancy and few researchers are incorporating these techniques into stem cell research other than for basic multiparameter characterization of cell lines. When employing retroviral constructs in reprogramming there is an additional concern over integration of viral DNA into the host genome and the integration at multiple sites, resulting in multiple clones from the reprogramming event (polyclonalism). It has been suggested that this, and other limitations, would interfere with early detection of fully reprogrammed cells by FACS alone. See, e.g., Chan et al., 2009, Nat Biotechnol 27: 1033-1037.

Thus, stem cells are an attractive source of cells for therapeutic applications, medical research, pharmaceutical testing and the like. However, there remains a longstanding need in the art for improved methods for producing and isolating iPSC cell lines in order to meet these and other needs.

### SUMMARY OF THE INVENTION

Accordingly, the invention provides a method for producing induced pluripotent stem cells (iPSC) from adult fibroblasts comprising the steps of:
(a) contacting adult fibroblasts with a reprogramming composition suitable for reprogramming the adult fibroblasts to iPSC under conditions effective for the reprogramming composition to penetrate the adult fibroblasts,
(b) culturing the contacted fibroblasts for a time period sufficient for the cells to be reprogrammed;
(c) sorting the cultured cells to select cells based upon their expression of the cell membrane surface markers CD13^{NEG} SSEA4^{POS} Tra-1-60^{POS}.
(d) identifying iPSC colonies from the sorted cells of (c).

The adults fibroblasts are preferably obtained by expanding fibroblasts from tissue biopsies, e.g., skin or other organs, by art standard methods. The reprogramming composition preferably comprises at least one expression vector expressing a transcription factor suitable for reprogramming the adult fibroblasts to iPSC. The expression vector expresses at least one transcription factor from Oct4, KLF4, Sox2, Lin28, Nanog, c-Myc, 1-Myc and combinations thereof, and is preferably the trascription factor is one of Oct4, KLF4, Sox, c-Myc and combinations thereof.

In a further embodiment, the inventive method is conducted with a reprogramming composition that also includes inhibitors of pathways such as the transforming growth factor-beta (TGFb) pathway, the MAPK/ERK pathway, the Glycogen synthase kinase 3 (GSK3) pathway, the histone deacetylase (HDAC) inhibitors, activators of 3 ′-phosphoinositide-dependent kinase-1 (PDK1), mitochondrial oxidation modulators, e.g., 2,4-dinitrophenol, lycolytic metabolism modulators, hypoxia-inducible factor-1 (HIF) pathway activators and combinations thereof.

Preferred inhibitors of the above-mentioned pathways preferably include, e.g., SB431542, A-83-01, PD0325901, CHIR99021, Parnate, PS48, sodium butyrate, valproic acid, 2,4-dinitrophenol, fructose 2,6-bisphosphate, oxalate, N-oxaloylglycine, Quercetin and combinations thereof.

In one embodiment of the invention, the identifying step (d) comprises visually screening and identifying those clones having the appearance of iPSC colonies. In further embodiments, the identifying step (d) comprises one or more of the following methods:

testing the sorted cells by flow cytometry (FC) or immunofluorescent (IF) microscopy to identify those cells with positive expression levels for a cell membrane surface markers selected from the group consisting of alkaline phosphatase, SSEA3, Tra-1-81, CD326 and combinations thereof.

testing the sorted cells by flow cytometry (FC) or immunofluorescent (IF) microscopy to identify those cells with positive expression levels for cell membrane surface markers selected from the group consisting of CD9, CD24, CD44, CD49c, CD49f, CD51/61, CD57, CD58, CD71, CD73, CD98, CD117, CD133, CD146, CD193, CD196, CD271, CD309, CD338 and combinations thereof.

testing the sorted cells by flow cytometry (FC) or immunofluorescent (IF) microscopy to identify those cells with positive expression levels for nuclear located transcription factors Oct 4, KLF4, Sox2, Nanog and combinations thereof.

Southern blotting of the sorted cells to identify unique clones based on patterns of viral DNA integration of Oct 4, KLF4, Sox2, or c-Myc transcription factors in the sorted cells.

In a still further embodiment, the testing step is conducted by quantitative real time PCR of the sorted cells to detect silencing of the retrovirally induced transcription factors Oct 4, KLF4, Sox2, or c-Myc and the endogenous expression of the Oct 4, KLF4, Sox2, or Nanog transcription factors in the sorted cells.

In yet a still further embodiment, the testing step is conducted by teratoma formation by the sorted cells in immunocompromised mice to confirm the ability of the sorted cells to form all three germ layers, or alternatively, the testing step is conducted by inducing the sorted cells to form Embryoid Bodies, thereby confirming the ability of the sorted cells to form all three germ layers. In a further alternative embodiment of the invention, the testing step is conducted by FACS enriching the CD13^{NEG} SSEA4^{POS} Tra-1-60^{POS} sorted cell populations into multiwell plates for high throughput derivation assays.

The inventive methods are readily employed wherein the adult fibroblasts are high passage fibroblasts, are obtained from biopsy tissue and/or are fibroblasts contaminated with known or unknown cell lines. Preferably, the fibroblasts are human fibroblasts, but the inventive methods are readily applied to nonhuman fibroblasts, as well.

While any suitable art-known expression vector(s) are employed by the inventive methods, preferred expression vectors include, e.g., a retrovirus, a lentivirus, an adenovirus, an adeno associated virus, a herpes virus, a Sindbis virus, a pox virus, a bacula virus, a bacterial phage, a Sendai virus and combinations thereof. More preferably, the Sendai virus is a nonreplicative virus.

Generally, step (a) is conducted by electroporation, chemical transfection or by means of cell penetrating proteins, of the adult fibroblasts. The chemical transfection is conducted, e.g., by means of a chemical transfecting agent selected from the group consisting of a cationic lipid, a polymer, calcium phosphate and combinations thereof. The cell penetrating protein is, for example, a TAT tagged protein and/or an arginine rich protein.

### BRIEF DESCRIPTION OF THE DRAWINGS

FIG. 1A Illustrates Fluorescence Activated Cell Sorting (FACS) of distinct populations of the manually derived 1018 clone. The CD13^{NEG}SSEA4^{POS}Tra-1-60^{NEG} and CD13^{NEG}SSEA4^{POS}Tra-1-60^{POS} populations were sorted onto MEF feeder layers and cultured for 20 days without manual cleaning of cultures prior to reanalysis by flow cytometery to detect retention of sorted surface markers. Boxed regions indicate distinct populations of interest and all percentages indicate proporation of cells contained within the boxed region compared to total cells in the sample. These data demonstrate that both the CD13^{NEG}SSEA4^{POS}Tra-1-60^{POS} and CD13^{NEG}SSEA4^{POS}Tra-1-60^{NEG} populations can be sorted to approximately 70% purity. The sorted CD13^{NEG}SSEA4^{POS}Tra-1-60^{POS} population retained higher proportion of reprogrammed fibroblasts (30% of total cells) than the CD13^{NEG}SSEA4^{POS}Tra-1-60^{NEG} population (14% of total cells) after 20 days of cultures Furthermore the sorted CD13^{NEG}SSEA4^{POS}Tra-1-60^{POS} population contained no unreprogrammed adult skin fibroblasts (CD13+)

FIG. 1B Illustrates time course analysis by Flow Cytometry (FC) of cell surface marker expression in human skin fibroblasts following reprogramming. Foreskin fibroblast 0825 and Adult skin fibroblasts 1018 and 1023 underwent 4 factor retroviral reprogramming and were analyzed by FC for the emergence of the CD13^{NEG}SSEA4^{POS}Tra-1-60^{POS} population at seven day intervals post infection. D₀, D₇, D₁₄ and D₂₁ represent days post infection 0, 7, 14 and 21, respectively. Day 0 indicates absence of pluripotent marker expression on surface skin fibroblasts which were uninfected but plated under the same conditions as the infected samples. All percentages indicate percent of total cells in the culture at the indicated time point post infection contained in the upper right quadrant of the plot.

FIG. 1C Illustrates colony formation when CD 13^{NEG}SSEA4^{POS} and CD13^{NEG}SSEA4^{POS}-Tra-1-60^{POS} populations were sorted onto MEF layers and imaged at 3 and 17 days post sort to assess colony formation. “dps” indicates days post sorting, “dpi” indicates days post-infection. Sorted CD13^{NEG}SSEA4^{POS}Tra-1-60^{POS} populations produce cleaner cultures of reprogrammed cells than the sorted CD13^{NEG}SSEA4^{POS} population and maintain that cleanliness by removing unreprogrammed, partially reprogrammed and transformed cells with overgrow the culture. Magnification 5×.

FIG. 2 Illustrates the normal Karyotype of the parent fibroblast and FACS and manually derived clones as performed by G-Banding.

FIG. 3A Illustrates photomicrographs of manually derived iPSC clones that were expanded on MEF feeder layers and stained for Tra-1-60 and Nanog expression indicating pluripotent status of colonies. Magnification 10×.

FIG. 3B Illustrates photomicrographs of FACS derived iPSC clones that were expanded on MEF feeder layers and stained for Tra-1-60 and Nanog expression indicating pluripotent status of colonies. Magnification 10×.

FIG. 3C illustrates the results of real time quantitative real time polymerase chain reaction (“qPCR”) that was performed on both the manually and FACS derived clones to demonstrate silencing of retroviral gene expression. The p# following the clone name indicates the passage number that the clone was maintained prior to analysis. 103hFB indicates uninfected fibroblast control cell line to show absence of virally induced transcription factor prior to infection. The 293 cell line indicates positive control cell line which stably expresses viral transfected transcription factors to show specificity of transcription factor primers and to normalize virally induced transcription factor in the reprogrammed fibroblast lines. The normalized expression levels for each transcription factor are indicated as Relative Expression on the Y Axis. The HUES62 line is an Embryonic Stem Cell (hES) line used as an additional negative control cell line to show lack of non-specific transcription primer binding in the assay.

FIG. 3D Illustrates the results of real time qPCR that was performed on both the manually and FACS derived clones to demonstrate activation of endogenous transcription factor expression. The pX following the clone name indicates the passage number that the clone was maintained prior to analysis. 103hFB indicates uninfected fibroblast control cell line to show absence of endogenous transcription factor expression prior to infection. The 293 cell line indicates positive control cell line which stably expresses viral transfected transcription factors to show absence of endogenous transcription factor expression prior to infection. The HUES62 line is an Embryonic Stem Cell (hES) line used as a positive control cell line for normalization of endogenous transcription primer expression in the assay. The normalized expression levels for each transcription factor are indicated as Relative Expression on the Y Axis.

FIG. 4A Illustrates photomicrographs of colonies of FACS derived AD iPSC line 7671 clone B that was expanded on MEF feeder layers and stained for SSEA4, Oct4 and Nanog expression indicating pluripotent status of colonies. Magnification 10×.

FIG. 4B illustrates real-time (RT) PCR of viral-specific transgene markers in clone 7671B using RNA from virally-infected 293 cells as a positive control.

FIG. 4C illustrates RT-PCR of endogenous stem cell genes, using RNA from human embryonic stem cells as a positive control, and virally-infected 293 as a negative control. Note that beta-2 microglobulin expression was used to normalize all data for D and E. The normalized expression levels for each transcription factor are indicated as Relative Expression on the Y Axis.

FIG. 5A Illustrates brightfield images of reprogrammed human fibroblast lines 0825, 1018 and 1023 at seven days post infection (dpi7) with either retroviral or Sendai virus. Visible colonies are absent in the retrovirally reprogrammed fibroblasts but are present in the fibroblasts reprogrammed using the Sendai virus (indicated by arrows). No Magnification

FIG. 5B Illustrates the proportions of CD13^{NEG}SSEA4^{POS}Tra-1-60^{POS} populations at dpi7 from the imaged cultures described in FIG. 5A. Very few CD13^{NEG}SSEA4^{POS}Tra-1-60^{POS} populations are present in the retrovirally reprogrammed fibroblasts but are present in higher proportions in the fibroblasts reprogrammed using the Sendai virus. Note that the 1023 line was more efficiently reprogrammed using the retroviral techniques. Magnification 5×.

### DETAILED DESCRIPTION OF THE INVENTION

Accordingly, improved methods for producing iPSC from differentiated adult cells are provided. Broadly, the invention provides methods for producing induced pluripotent stem cells (iPSC) from adult fibroblasts by:

(a) reprogramming adult fibroblasts by contacting the adult fibroblasts with a reprogramming composition suitable for reprogramming the adult fibroblasts to iPSC, under conditions effective for the reprogramming composition to penetrate the adult fibroblasts;

(b) culturing the contacted fibroblasts for a time period sufficient for the cells to be reprogrammed;

(c) sorting the cultured cells to select for reprogrammed cells based upon their expression of the cell membrane surface markers CD13^{NEG} SSEA4^{POS} Tra-1-60^{POS} and

(d) identifying iPSC colonies from the sorted cells of (c).

In an alternative embodiment, the invention provides methods for producing induced pluripotent stem cells (iPSC) from adult fibroblasts by:

(a) contacting the adult fibroblasts with a reprogramming composition suitable for reprogramming the adult fibroblasts to iPSC, under conditions effective for the reprogramming composition to penetrate the adult fibroblasts;

(b) culturing the contacted fibroblasts for a time period sufficient for the cells to be reprogrammed;

(c) sorting the cultured cells to select for reprogrammed cells based upon their expression of the cell membrane surface markers CD13^{NEG} SSEA4^{POS} Tra-1-60^{POS} and

(d) identifying iPSC colonies from the sorted cells of (c).

As used herein “adult” means post-fetal, i.e., an organism from the neonate stage through the end of life.

As used herein, the term “induced pluripotent stem cells” or iPSC means that the stem cells are produced from differentiated adult cells that have been induced or changed, i.e., reprogrammed, into cells capable of differentiating into tissues of all three germ or dermal layers: mesoderm, endoderm, and ectoderm. The iPSCs produced do not refer to cells as they are found in the nature.

In a preferred embodiment the methods of the invention include steps for enriching early reprogrammed fibroblasts expressing the combination of CD13^{NEG} SSEA4^{POS} Tra-1-60^{POS} surface markers using FACS. The inventive methods both enrich the cells of interest and remove partially reprogrammed and adult fibroblasts and effectively reduce the time, labor, and resources required to generate stable, monoclonal iPSClines. The inventive methods are also effective in generating clones from fibroblast lines that exhibit resistance to current reprogramming technology. The inventive methods can also be applied to fibroblasts from normal and disease specific samples reprogrammed under multiple techniques, including retroviral and Sendai viral systems.

In a further preferred embodiment, the inventive methods can also be used to obtain cell populations enriched in fully reprogrammed cells, from among cells that have undergone differentiation in established iPSC cell lines that were cultured under both murine embryonic fibroblast (MEF) feeder layer, as well as feeder free conditions. The inventive methods further enable the live sorting of defined subpopulations of fully-reprogrammed, or differentiated, iPSC cells into 96 well plates for use in high throughput screening campaigns.

Methods for transfecting and transforming or reprogramming adult cells to form iPSC lines are generally known, e.g., Takahashi et al., 2007 Cell, 131: 861-872, 2007, Yu et al., 2007, Science, vol. 318, pp. 1917-1920. iPSC are induced from somatic cells by introducing and expressing a combination of specific transcription factors, e.g., a combination of Oct3/4, Sox2, Klf4 and c-Myc genes. Others have demonstrated that other transcription factors may be employed in transforming or reprogramming adult cells. These other transcription factors include, e.g., Lin28, Nanog, hTert and SV40 large T antigen as described, for example, by Takahashi et al., 2006 Cell, 126: 663-676 and Huiqun Yin, et al. 2009, Front. Agric. China 3(2): 199-208, incorporated by reference herein.

It has also been shown that a single transcription factor may be employed in reprogramming adult fibroblasts to iPSC with the addition of certain other small molecule pathway inhibitors. Such pathway inhibitors include e.g., the transforming growth factor-beta (TGFb) pathway inhibitors, SB431542 (4-[4-(1,3-benzodioxol-5-yl)-5-(2-pyridinyl)-1H-imidazol-2-yl]-benzamide), and A-83-01 [3-(6-Methyl-2-pyridinyl)-N-phenyl-4-(4-quinolinyl)-1H-pyrazole-1-carbothioamide], the extracellular signal-regulated kinases (ERK) and microtubule-associated protein kinase (MAPK/ERK) pathway inhibitor PD0325901 (N-[(2R)-2,3-dihydroxypropoxy]-3,4-difluoro-2-[(2-fluoro-4-iodophenyl)amino]-benzamide), the GSK3 inhibitor CHIR99021 [6-((2-((4-(2,4-Dichlorophenyl)-5-(4-methyl-1H-imidazol-2-yl)pyrimidin-2-yl)amino)ethyl)amino)nicotinonitrile] which activates activates Wnt signalling by stabilizing beta-catenin, the lysine-specific demethylase1 Parnate (a/k/a tranylcypromine), the small molecule activator of 3′-phosphoinositide-dependent kinase-1 (PDK1) PS48 [(2Z)-5-(4-Chlorophenyl)-3-phenyl-2-pentenoic acid], the histone deacetylase (HDAC) inhibitors sodium butyrate and valproic acid, small molecules that modulate mitochondrial oxidation (e.g., 2,4-dinitrophenol), glycolytic metabolism (fructose 2,6-bisphosphate and oxalate), HIF pathway activation (N-oxaloylglycine and Quercetin) Zhu et al., 2010, Cell Stem Cell 7: 651-655, incorporated by reference herein it its entirety. Zhu et al showed that Oct4 combined with Parnate and CHIR99021 was sufficient to reprogram adult human epidermal keratinocytes.

Although individual protocols differ, a general reprogramming protocol consists of expanding fibroblasts from tissue samples, e.g., skin biopsies and infecting them, ie., transfecting, with e.g., expression vectors, such as viral constructs containing transcripts for pluripotent transcription factors. The fibroblasts are obtained by art-known methods, e.g., by mechanically disrupting the tissue followed by enzymatic dissociation to release the fibroblasts, and culturing the fibroblasts by art-known methods, e.g., as described by Dimos et. al., 2008, Science Vol. 321 (5893): 1218-1221.

Transfection of the fibroblasts with an expression vector is carried out according to instructions provided with the desired vector. After a time ranging from about 3 to about 7 days post-transfection, the cells are dissociated and contacted with fluorescent tagged antibodies raised against the CD13^{NEG}, SSEA4^{POS} and Tra-1-60^{POS} surface markers. The dissociated and antibody-labeled cells are then resuspended in a phosphate buffered saline solution and loaded onto the FACS machine. Surface marker positive cells are sorted by tag color or absence thereof directly into sterile tubes containing tissue culture media or multiwell (6-96 well) tissue culture plates coated with MEFs or cell free biological matrices and cultured until formation of visible colonies occurs.

Colonies are then further confirmed as iPSC by light microscopic inspection of the resulting clones or optionally by microscopic fluorescence inspection of clones labeled with fluorescent tagged antibodies. Optionally, in certain embodiments, one or more of the vectors also insert a green flourescence protein (GFP) expression marker, for convenience in sorting and identification. Several individual colonies possesing morphological characteristics consistent with pluripotent ES lines are plucked from cultures and expanded individually to form monoclonal cultures.

In one preferred embodiment of the inventive method, the sorted cells are subjected to genetic analysis to provide early confirmation and identification of iPSC cells. Preferably, the genetic analysis is conducted by Southern blot, but other art-known methods may be employed which include but are not limited to MicroArray, Nano String, quantitative real time PCR (qPCR), immunofluorescence microscopy, flow cytometry. Detection of enzymatic activity of alkaline phosphatase, positive expression of the cell membrane surface markers SSEA3, SSEA4, Tra-1-60, Tra-1-81 and the expression of the KLF4, Oct3/4, Nanog, Sox2 transcription factors in reprogrammed human fibroblasts confirms that a clone is an iPSC. Preferably, all of the markers are present.

Any art-known transfection vector may be employed, including, e.g., an RNA such as mRNA, microRNA, siRNA, antisense RNA and combinations thereof. Other expression vectors that may be employed include, e.g., a retrovirus, a lentivirus, an adenovirus, an adeno associated virus, a herpes virus, a Sindbis virus, a pox virus, a bacula virus, a bacterial phage, a Sendai virus and combinations thereof. Preferably, an employed vector is a non-replicative vector such as, e.g., Sendai virus vectors engineered to be nonreplicative. The preferred Sendai virus vector, while incapable of replication, remains capable of productive expression of nucleic acids encoding protein(s) carried by the vector, thereby preventing any potential uncontrolled spread to other cells or within the body of a vaccinee. This type of Sendai vector is commercially available as a CytoTune™-iPS Sendai viral vector kit (DNAVEC, DV-0301).

Any art known transfection method may be employed to insert such vectors into the adult fibroblasts, including, e.g., electroporation, gene gun, and the like. Chemical transfection is optionally conducted by means of a transfecting agent e.g., a polymer, calcium phosphate, a cationic lipid, e.g., for lipofection, and the like. Cell penetrating peptides are also optionally employed to carry vectors or other agents into the adult fibroblast cells. In brief, cell penetrating peptides include those derived from proteins, e.g., protein transduction domains and/or amphipathic peptides, that can carry vectors or other agents into the cell include peptides. The subject of cell penetrating peptides has been reviewed, e.g., by Heitz et al., 2009 British Journal of Pharmacology, 157: 195-206, incorporated by reference herein in its entirety. Other cell penetrating peptides are art-known, and are disclosed by Heitz, Id. Other cell penetrating technologies including, e.g., liposomes and nanoparticles, are also contemplated to be employed in the methods of the present invention. Liposomes and nanoparticles are also described by Heitz, Id.

Antibodies are employed in order to tag the transformed cells for FACS sorting. Four antibodies against stem cell specific surface proteins are commonly used to identify and characterize human pluripotent stem cell populations; SSEA3, SSEA4, Tra-1-60 and Tra-1-81. The Stage Specific Embryonic Antigens 3 and 4 (SSEA3 and SSEA4) are two monoclonal antibodies which recognize sequential regions of a ganglioside present on human 2102Ep cells (Henderson et al., 2002 Stem Cells 20: 329-337; Kannagi et al., 1983, Embo J 2: 2355-2361). The Tra-1-60 and Tra-1-81 antibodies were originally raised against human embryonal carcinoma (EC) cells (PW et al., 1984, Hybridoma 3: 347-361) and have been shown to specifically recognize a carbohydrate epitope on a keratan sulfated glycoprotein identified as podocalyxin, a member of the CD34-related family of sialomucins (Badcock et al., 1999, Cancer Research 59: 4715-4719; Nielsen et al., 2007, PLoS ONE 2: e237; Schopperle and DeWolf, 2007, Stem Cells 25: 723-730). Several other surface markers have been shown to be expressed on ES cells and include CD326 or EpCam (Sundberg et al., 2009, Stem Cell Res 2: 113-124), CD24 (Heat Stable Antigen) and CD133 (Barraud et al., 2007, Journal of Neuroscience Research 85, 250-259) (Gang et al., 2007, Blood 109: 1743-1751). Chan et al., 2009, Id. reported that the identification of bona fide IPSc from fibroblasts undergoing reprogramming via four factor retro viral transduction can be achieved via live cell imaging and by the observation, over time, that fibroblasts lose expression of the cell surface markers CD13 and D7Fib, and gain expression of the pluripotent stem cell markers SSEA4 and Tra-1-60 (Chan et al., 2009, Id.).

Also contemplated to be within the scope of the invention are compositions comprising iPSCs, e.g., pharmaceutical compositions comprising effective amounts of iPSCs prepared by the inventive methods.

The invention further relates to methods of treating a disease or disorder in an animal or person in need thereof by administering the iPSCs, e.g., methods of treatment and/or tissue/organ repair by administering iPSCs or differentiated cells derived therefrom. Appropriate differentiated cells (of ectodermal, mesodermal or endodermal lineage) may be derived from iPSCs produced by the inventive methods. The mode of administration can be determined by a person of skill in the art depending on the type of organ/injury to be treated. For example, iPSCs or differentiated cells derived therefrom, may be administered by injection (as a suspension) or implanted on a biodegradable matrix.

In another embodiment, the iPSCs produced by the inventive methods may be used as a vehicle for introducing genes to correct genetic defects, such as osteogenesis imperfecta, diabetes mellitus, neurodegenerative diseases such as, for instance, Alzheimer's disease, Parkinson's disease, the various motor neuron diseases (MND), e.g., amyotrophic lateral sclerosis (ALS), primary lateral sclerosis (PLS), progressive muscular atrophy (PMA) and the like.

iPSCs produced by the inventive methods may also be employed to provide specific cell types cells for biomedical research, as well as directly or as precursors to produce specific cell types for cell-based assays, e.g., for cell toxicity studies (to test the effect of test compounds on cell toxicity), to test teratogenic or carcinogenic effects of test compounds by treating the cells with the compound and observing and/or recording the compound's effects on the cells, e.g. effect on cellular differentiation.

The present invention may be better understood by reference to the following non-limiting Examples. The following examples are presented in order to more fully illustrate the preferred embodiments of the invention. They should in no way be construed, however, as limiting the broad scope of the invention.

### Example 1

### Rapid Production of iPS Cells and Colonies

A. Cell Lines

The 0819 and 0825 fibroblast lines were derived from discarded foreskin tissue provided from a cell bank under a notice of Investigational Review Board (IRB) exemption. The 1018 fibroblast line was derived from an upper aim skin biopsy taken from a 32yo F with Type I Diabetes age of onset 10. The 1023 fibroblast line was derived from an upper arm skin biopsy (described below) taken from a 23yo M and is considered a healthy control. Fibroblasts derived from Alzheimer's Disease (AD) patients were obtained through Coriell Institute for Medical Research Cell Repository (website is located at CCR dot CORIELL dot ORG). Live cell cultures of all parent fibroblast and reprogrammed lines were sent to Cell Line Genetics (website is located at WWW dot CLGENETICS dot COM) for cytogenetic analysis by 20 G-banded metaphase cells to determine Karyotype and DNA fingerprinting by STR analysis using the Powerplex® 16 kit from Promega.

B. Fibroblast Cell Culture

Skin fibroblasts were derived from explants of 3-mm dermal biopsies which were minced with scalpels and placed into 60-min tissue culture dish under a sterile coverslip held down by sterilized silicon grease. Fibroblast medium (Dulbecco's modified Eagle's medium (DMEM) Invitrogen 11885092) supplemented with 10% fetal bovine serum (FBS) (various suppliers), Glutamax™ (Gibco 35050079), and penicillin/streptomycin (Invitrogen 15070063) was added to completely immerse the coverslip, and dishes were incubated at 37° C. in a humidified incubator (5% CO2). Media was changed every 5 days without disturbing the coverslip. Fibroblasts grew out of the tissue fragments, and when sufficiently numerous, cells were trypsinized and expanded.

C. Fibroblast Reprogramming

Fibroblasts were reprogrammed using a combination of OCT4, SOX2, cMYC, and KLF4 containing Vesicular Stomatitis Virus G (VSVG)—coated retroviruses (Harvard Gene Therapy Initiative) or the CytoTune™-iPS Sendai viral vector kit (DNAVEC, DV-0301) according to manufacturers' recommended protocol. Briefly, fibroblasts were thawed or split and plated on gelatin coated (Millipore ES-006-B) 6-well polystyrene TC plate and allowed to recover for four hours.

Fibroblasts reprogrammed using VSVG-coated retroviruses were plated at 10,000 cells per well of and infected in 1 ml of Human Embryonic Stem Cell Media (HuESM). On day 1 they were supplemented with 1 ml of fibroblast medium. The media was changed on day 2 to HuESM+10 ng/ml basic fibroblast growth media (bFGF) and/or +SB431542 (2 μM) (Stemgent, Cat#04-0010)+PD0325901 (0.5 μM) (Stemgent, Cat#04-0006)+Thiazovivin (0.5 μM) (Stemgent, Cat#04-0017) everyday following day 2. Fibroblasts reprogrammed using Sendai viruses were performed on 5*10⁵ fibroblasts for 2 days at a Muliplicity of Infection equal to 3 (MOI 3) Subsequently, the cells were fed every day with HuESM+10 ng/ml basic fibroblast growth media (bFGF) and/or +SB431542 (2 μM)(Stemgent, Cat#04-0010)+PD0325901 (0.5 μM) (Stemgent, Cat#04-0006)+Thiazovivin (0.5 μM) (Stemgent, Cat#04-0017) human ES cell media (knockout DMEM supplemented with 20% knockout serum replacement (Invitrogen 10828028), 10 ng/mL bFGF (Invitrogen 13256029), nonessential amino acids (Invitrogen 11140050), β-mercaptoethanol (Invitrogen 21985023), L-glutamine, and penicillin/streptomycin (Invitrogen 15070063).

On day 7 cells were enzymatically either passaged on to irradiated Murine Embryonic Fibroblasts (MEF) (Globalstem GSC-6001G) or Matrigel (BD Biosciences) feeder plates on HuESM at a density of 20,000 cells per well of a 6-well plate or subjected to FACS. Skin and foreskin iPS lines were cultured in human ES media on MEFs or Matrigel and passaged enzymatically using either Dispase® (GIBCO 17105041) and/or Accutase® (Sigma-Aldrich A6964).

D. Fluorescent Activated Cell Sorting (FACS) of Reprogrammed Fibroblasts

Cells were harvested by treatment with Dispase® (1 mg/ml in HuESM) for 5 minutes then dissociated with Acutase® for 10 minutes at 37° C. in a humidified incubator (5% CO₂) and then washed with 4 ml of HuESM. Gentle trituration was used, and cells were filtered through cell strainer caps to obtain a single cell suspension prior to incubation with fluorescent antibody cocktail (15 minutes, room temperature protected from light) composed of 1 μl each CD13 PE, SSEA4 AlexaFluor647®, and Tra-1-60 AlexaFluor488® (See Table 1 for conjugated antibody information) in a total volume of 100 μL of iPS staining buffer Dulbecco's Phosphate-Buffered Saline (DPBS) (Invitrogen 14190250), 0.5% bovine serum albumen (BSA) Fraction V (Invitrogen 15260037), 100 U/ml Penicillin Streptomycin (Invitrogen 15070063), 2 mM EDTA (Invitrogen 15575038), and 20 mM Glucose (Sigma G6152) filtered through a 0.22 μm vacuum filter. Stained cells were washed once with 1 ml iPS staining buffer and sorted immediately on a 5 laser BDbiosciences ARIA-IIu™ SOU Cell Sorter configured with a 100 μm ceramic nozzle and operating at 20 psi sheath fluid pressure.

Some experiments included the following monoclonal antibodies: D7Fib PE (AbDSerotec MCA1399PET), SSEA3 efluor® 605NC (ebiosciences 93-8833-41) or CD326/EpCAM PerCP Cy5.5 (BD 347199) in the antibody cocktails to confirm the pluripotent status of the reprogrammed cells.

Target cell populations were sorted directly onto MEF feeders (ARIA plate holder at 3°7 C.) at 2000-50,000 cells per well of a 6-well plate with HuESM+20 μM y-2′7632 (ROCK inhibitor (Calbiochem, Cat#688000)). ROCK inhibitor was maintained for 2 days after cell sorting and media was changed to either regular HuESM or HuESM with 10 ng bFGF ALK5 inhibitor SB431542 (2 μM) [Stemgent, Cat#04-0010], MEK inhibitor PD0325901 (0.5 μM) [Stemgent, Cat#04-0006] and Thiazovivin (0.5 μM) [Stemgent, Cat#04-0017] every day. Colonies were picked 3-5 days after sorting, whether the sort was conducted 7 days post infection or later.

E. Lyoplate™ Characterization

CD13^{NEG}SSEA4^{POS}Tra-1-60^{POS} populations or fibroblasts from the 0825, 1018 or 1023 lines were sorted onto Matigel™ coated (250 μl in 25 ml TESR® media; Stemcell Technologies 5850) 96 well black imaging plates (BD 353319) at 10⁵ cells/well and were cultured to 90% confluency prior to fixation with 4% paraformaldahyde in phosphate buffer (4% PFA) (Poly Scientific S2303) for 10 min at room temperature. The antibodies from one BD Lyoplate™ Human Cell Surface Marker Screening Panel (BD 560747) were prepared according to manufacturer's specifications. Primary antibodies were added to the imaging plates at (5 μL/well, 0.1 μg/well) and incubated overnight at +4° C. Secondary antibodies were added at 1:1000 for in PBS containing DAPI (Invitrogen D21490) for 1 hr at room temperature (RT) protected from light prior to one final wash and resuspension in final volume of 100 μl PBS. The plates were sealed and stored at +4° C. prior to imaging. All fixation, reagent addition, and well washing procedures were performed using an Agilent Bravo Liquid Handler and a Biotek EL406 Plate washer. Cell surface markers evaluated to be positive staining hits by lyoplate assay were validated by flow cytometry by using cocktails of directly conjugated antibodies (see Table 1, below) to stain single cell suspensions of live cells prepared as previously described in Section D.

TABLE 1
Conjugated Antibodies for Flow Cytometry

| Marker | Color | Company | Cat # |
|---|---|---|---|
| SSEA-4 | V450 | BD | 561156 |
| SSEA4 | AlexaFluor | BD | 560173 |
| | 647 | | |
| Tra-1-60 | AlexaFluor | BD | 560173 |
| | 488 | | |
| Alkaline | AlexaFluor | BD | 561500 |
| Phosphatase | 647 | | |
| CD133 | APC | Miltenyi | 130-080-801 |
| | | biotec | |
| CD326 | APC | BD | 347200 |
| CD13 | PE | BD | 555394 |
| D7Fib | PE | AbDSerotec | MCA1399PE |
| SSEA3 | PE | BD | 560237 |
| Tra-1-81 | PE | BD | 560161 |
| CD9 | PE | BD | 555372 |
| CD24 | PE | BD | 555428 |
| CD44 | V450 | BD | 561292 |
| CD49c | PE | BD | 556025 |
| CD49f | PE | BD | 555736 |
| CD51/61 | PE | BD | 550037 |
| CD57 | PE | BD | 560844 |
| CD58 | PE | BD | 555921 |
| CD71 | PE | BD | 555537 |
| CD73 | PE | BD | 550257 |
| CD98 | PE | BD | 556077 |
| CD117 | PE | BD | 340529 |
| CD146 | PE | BD | 550315 |
| CD164 | PE | BD | 551298 |
| CD196 | PE | BD | 559562 |
| CD271 | PE | BD | 557196 |
| CD309 | PE | BD | 560494 |
| CD338 | PE | BD | 561180 |

### Immunofluorescence and Microscopy

Unconjugated antibodies used in the microscopy experiments are provided in Table 2 below.

TABLE 2
Primary Antibodies for Immunofluorescence

| Antibody | Company | Catalog # |
|---|---|---|
| Oct4 | Stemgent | 09-0023 |
| Sox2 | Stemgent | 09-0024 |
| Tra-1-60 | Millipore | MAB4381 |
| SSEA4 | R&D Systems | MAB1435 |
| Nanog | R&D Systems | AF1997 |
| SSEA3 | R&D Systems | MAB1434 |

G. RT/PCR

Total RNA was isolated using RNAeasy kit (QIAGEN, Cat. No. 74104) from duplicate or triplicate samples. cDNA synthesis was performed on 1 μg RNA with SuperScript™ III First-Strand system (Invitrogen, Cat. No. 18080-051) and Oligo (dT) primers. The resulting cDNA was diluted to a final volume of 200 μl and 1 μl of the cDNA dilution and 500 nM of forward and reverse primers are used for each 10 μl PCR reaction. Quantitative real-time PCR was performed using the LightCycler® SYBR Green Master kit (Roche, Cat. No. 04707516001) and Mx3000p QPCR system (Stratagene). The PCT primers are described in Table 3, below.

TABLE 3

| | FORWARD | REVERSE |
|---|---|---|
| GENE | PRIMER 5′-3′ | PRIMER 5′-3′ |
| Oct 4 | CCCCAGGGCCCCATT | GGCACAAACTCCAGG |
| (endogenous) | TTGGTACC | TTTTC |
| | (SEQ ID NO: 1) | (SEQ ID NO: 2) |
| Sox2 | ACACTGCCCCTCTCA | GGGTTTTCTCCATGC |
| (endogenous) | CACAT | TGTTTCT |
| | (SEQ ID NO: 3) | (SEQ ID NO: 4) |
| Klf4 | ACCCACACAGGTGAGA | GTTGGGAACTTGACC |
| (endogenous) | AACCTT | ATGATTG |
| | (SEQ ID NO: 5) | (SEQ ID NO: 6) |
| C-Myc | AGCAGAGGAGCAAAAG | CCAAAGTCCAATTTG |
| (endogenous) | CTCATT | AGGCAGT |
| | (SEQ ID NO: 7) | (SEQ ID NO: 8) |
| Oct4 | CCCCAGGGCCCCATTT | AACCTACAGGTGGGG |
| (transgene) | TGGTACC | TCTTTCA |
| | (SEQ ID NO: 9) | (SEQ ID NO: 10) |
| Sox2 | ACACTGCCCCTCTCAC | AACCTACAGGTGGGG |
| (transgene) | ACAT | TCTTTCA |
| | (SEQ ID NO: 11) | (SEQ ID NO: 12) |
| Klf4 | GACCACCTCGCCTTAC | AACCTACAGGTGGGG |
| (transgene) | ACAT | TCTTTCA |
| | (SEQ ID NO: 13) | (SEQ ID NO: 14) |
| C-Myc | AGCAGAGGAGCAAAAG | AACCTACAGGTGGGG |
| (transgene) | CTCATT | TCTTTCA |
| | (SEQ ID NO: 15) | (SEQ ID NO: 16) |
| B2M | TAGCTGTGCTCGGGCT | TCTCTGCTGGATGAC |
| | ACT | GCG |
| | (SEQ ID NO: 17) | (SEQ ID NO: 18) |

H. Teratoma Assay

Single wells of a standard 6 well tissue culture plate containing the manually derived 1023A line (passage p13) and 1023C line passage or FACS derived 1023D2 (passage 12) at 70% confluency are dissociated using Dispase (Gibco #17105-041) for 15-20 minutes at 37° C. and 5% CO₂ to produce small clumps containing approximately 100-200 iPS cells/clump. iPS containing chimps are resuspended in 100 ml of HuESM and mixed with an equal volume of thawed Matrigel™ and transfered to ice cold cryotubes (Nunc 377267). Cell mixtures are held on ice until injecting into the hindlimb muscle of NOD-SCID Il2rg-null mice (Jackson Laboratory Stock No 005557) that are pre-injected intraperitoneal (ip) with Carprofen (Pfizer 141-199) at 5 mg/kg body weight. Teratomas are allowed to grow for 6-8 weeks prior to recovery by dissection and fixation in 4% PFA overnight at +4° C. Fixed tissue are sent to the Columbia University Medical Center histology service where they are processed according to standard procedures for paraffin embedding, sectioned onto glass microscope slides and stained with hematoxylin and eosin (H&E). Histological analysis showed that teratomas were consisting of a variety of all three germ layer tissues, including gut-like epithelial tissues (endoderm), muscle (mesoderm), cartilage (mesoderm), neural tissues (ectoderm) and retina pigment epithelium (ectoderm).

I. Functional Differentiation Assays

CD13^{NEG}SSEA4^{POS}Tra-1-60^{POS} populations from passage 9 of the 1023D2 clone were FACS into 96 well plates at high (10⁴/well) or low (2*10³/well) densities and treated with mTeSR®1 complete media (Stemcell Technologies 5850) or Custom mTeSR®, Five Factor Free (Stemcell Technologies 5898) alone or in combination with Recombinant Bone Morphogenetic Protein 4 (BMP4) (R&D Systems 314BP/CF) @ 200 ng/ml, or 10 μM SB431542 (Stemgent Stemolecule™ SB431542 04-0010)+LDN193189 (Stemgent Stemolecule™ LDN193189 04-0074) 250 nM. Cells were incubated for 7 days then fixed 4% PFA for 10 min at RT. Fixed cells were incubated with primary antibodies overnight at +4C, for 1 hr RT in dark with secondaries. One high resolution field per well at 10× magnification was acquired using the Cellomics Arrayscan HCS and analyzed using the Cellomics Compartmental Analysis BioApplication.

J. Embryoid Body Formation Embryoid bodies (EB) were formed by placing clumps of hiPS in 96-well non-tissue culture treated V-bottom plates (Evergreen 222-8031-01V) and cultured for 3-4 weeks in HuESM without bFGF every 2-4 days. EB were fixed in 4% PFA for 30 minutes and prepared for histology sectioning by incubating overnight in graded concentrations of sucrose. EB sections were stained to detect the three germ layers using the following antibodies. Mesoderm Brachyury (Santa Cruz sc-20109), Muscle Actin (MF20) DSHB MF20. Endoderm AFP (DAKO A0502) HNF3b (Santa Cruz sc-6554) Ectoderm NFH (Sternberger SMI32), beta III Tubulin (Neuromics CH23005).

K. Image Acquisition and Analysis

The Cellomics Arrayscan HCS Reader (Thermo Scientific) was used to acquire 1-10 high resolution images per well from each 96 well plate ranging from 5-20× magnification dependent upon the number of colonies present in each well. Images were analyzed using the Cellomics Compartmental Analysis BioApplication which measured mean ring intensity in the Cy5 channel using (Filter Set) Positive staining for surface markers was verified by multi-color flow cytometry.

L. Southern Blot Protocol

Probes for human Oct4, Sox2, and KLF4 were generated by PCR using the digoxigenin (DIG) probe synthesis kit. Genomic DNA was isolated from HUESCs, parent fibroblast cells, and iPS cells using the Qiagen DNA Mini kit, and 5-10 μg of DNA were digested overnight with BglII to generate a single cut in the integrated viral backbone on all transgenes used. Digests were run along with a DIG-incorporated ladder on a 0.8% agarose gel (no EthBr), which was then denatured with 0.5% NaOH followed by neutralization. The gel was then transferred to nylon membranes by utilization of overnight capillary transfer.

On Day 3, wet membranes were crosslinked with 120 mJ UV (HL-2000 Hybrilinker, WP) and allowed to dry. Membranes were then pre-hybridized with DIG easy buffer for at least 1 hour at 55° C., then put in appropriate probe overnight at 55° C. On Day 4, membranes were washed appropriately using the DIG wash and block kit, blocked for at least 1 hour, and treated with anti-DIG antibody for 30′. Membranes were then washed appropriately using DIG Block and buffer kit reagents, and treated with CDP-Star reagent to detect DIG-incorporated bands.

Blots were stripped and re-probed according to the manufacturer's instruction. All reagents were from Roche, and used as per the manufacturer's suggestion: PCR DIG probe synthesis kit, CDP-Star, DIG Easy Hyb, DIG wash and block buffer set, anti-DIG-AP antibody, DIG DNA molecular marker, and positively charged nylon membranes.

### Primers

The Southern blot primers are described by Table 4, below.

TABLE 4

| | FORWARD | REVERSE |
|---|---|---|
| GENE | PRIMER 5′-3′ | PRIMER 5′-3′ |
| Oct 4 | GAGAAGGAGAAGCT | GTGAAGTGAGGGCT |
| (endogenous) | GGAGCA | CCCATA |
| | (SEQ ID NO: 19) | (SEQ ID NO: 20) |
| Sox2 | AGAACCCCAAGATGC | TGGAGTGGGAGGAAG |
| (endogenous) | ACAAC | AGGTA |
| | (SEQ ID NO: 21) | (SEQ ID NO: 22) |
| Klf4 | ACCTGGCGAGTCTGA | TCTTCATGTGTAAGG |
| (endogenous) | CATGG | CGAGGTGG. |
| | (SEQ ID NO: 23) | (SEQ ID NO: 24) |

M. Results

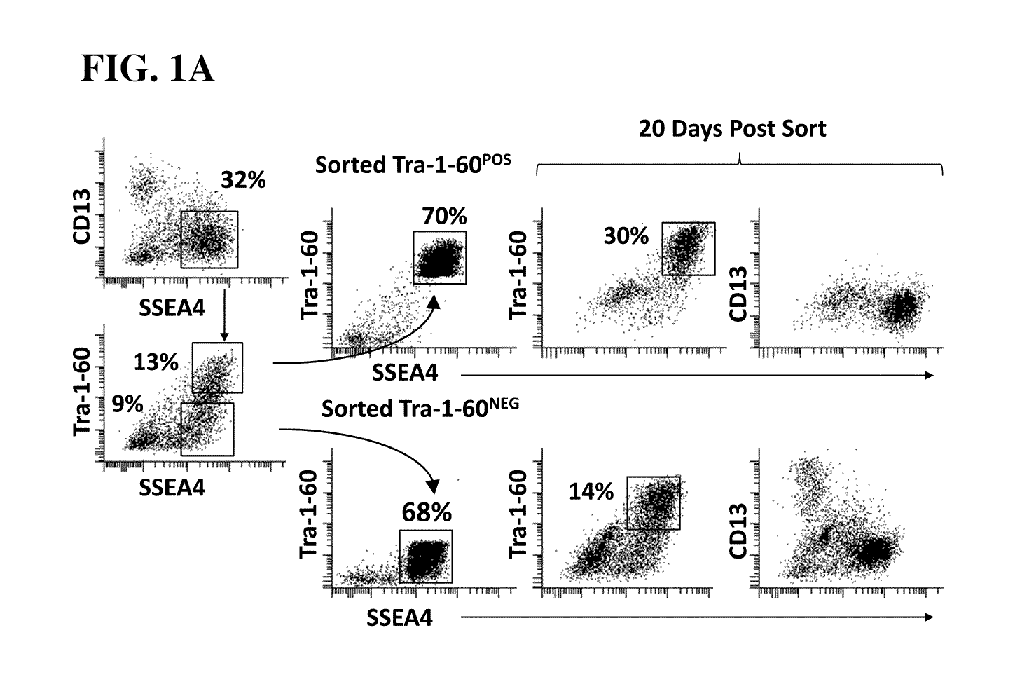

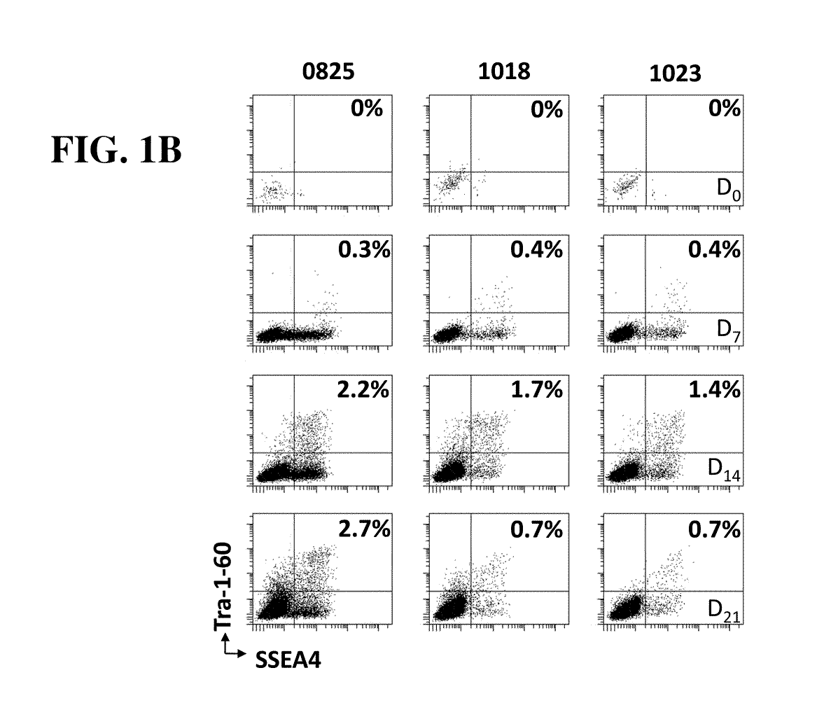

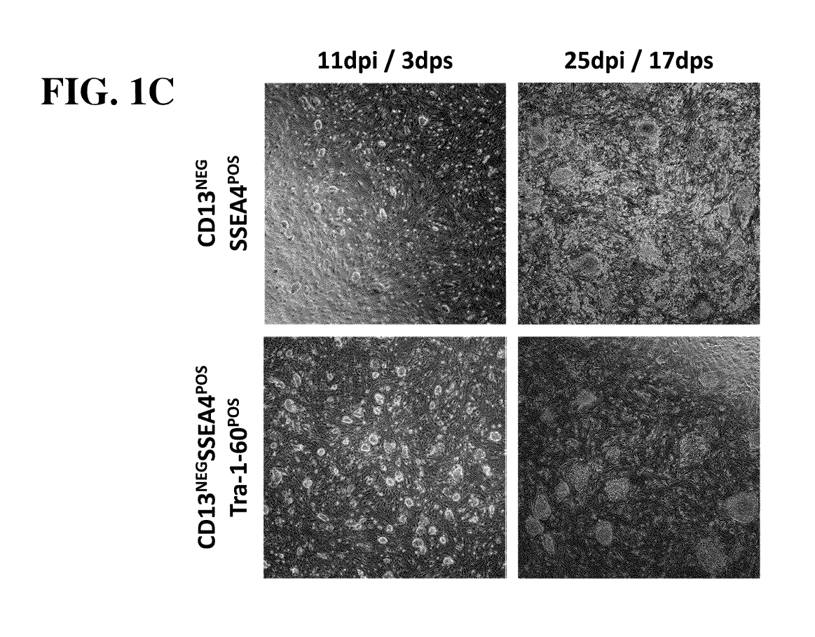

To assess the ability of FACS to live sort reprogrammed fibroblasts a stable clone of the 1018 T1 D line was selected. This line had previously been established by manual picking and maintenance and FACS was used to enrich for the CD13^{NEG}SSEA4^{POS}Tra-1-60^{NEG} and the CD13^{NEG}SSEA4^{POS}Tra-1-60^{POS} populations in this line. Following 20 days of culture on MEFs the cultures were dissociated and surface marker expression was measured by flow cytometry (FC) (FIG. 1A). Adult fibroblasts expressing CD13^{POS} and CD13^{NEG}SSEA4^{POS}Tra-1-60^{POS} populations were present in the wells containing sorted CD13^{NEG}SSEA4^{POS}Tra-1-60^{NEG} cells, indicating that a proportion of Tra-1-60^{NEG} cells were still undergoing reprogramming.

The presence of adult CD13^{POS} fibroblasts may have been due to carryover in the form of doublets (target cells plus nontarget cells) resulting from incomplete dissociation. Alternatively, partially transformed cells may retain the ability to revert back to a fibroblast like state. As expected, the well containing sorted CD13^{NEG}SSEA4^{POS}Tra-1-60^{POS} populations contained lower proportions of differentiated cells and very few adult fibroblasts expressing CD13^{POS}.

These data demonstrate the ability of FACS to simultaneously deplete the adult fibroblasts from cultures and enrich reprogrammed cells.

To investigate the kinetics of fibroblast reprogramming using the retroviral vector, four factor system and determine the earliest time point post infection at which reprogrammed fibroblasts from could be successfully sorted from cultures of adult skin fibroblasts, the emergence of a population of cells negatively expressing the adult fibroblast marker CD13 and positively expressing the pluripotent surface markers SSEA4 and Tra-1-60 (CD13^{NEG}SSEA4^{POS}Tra-1-60^{POS}) was measured at 7 days intervals post infection (dpi) using flow cytometry. Previous time course expression analyses (Data Not Shown) carried out on foreskin fibroblasts for >30 dpi suggested that small number of SSEA4^{POS}Tra-1-60^{POS} are present in cultures as early at dpi7, and increase in proportion by dpi21, but decrease at later time points as uninfected and transformed fibroblasts take over the culture.

The previous analyses was extended to include a foreskin (0825), a control (1023) and Type 1 diabetes (T1D) adult skin fibroblast line (1018), in order to investigate variability in reprogramming kinetics between tissue samples and healthy and disease types. As in previous experiments a similar trend was observed in the emergence of SSEA4^{POS}Tra-1-60^{POS} cells in all cultures at dpi7, which increased in proportion with partially transformed cells, and decreased at later time points as uninfected and transformed fibroblasts took over the culture. (FIG. 1B). The double positive CD13^{NEG}SSEA4^{POS}Tra-1-60^{POS} population continued to increase in the foreskin line but decreased in both adult fibroblast lines, although they contained less transformed cells.

Based on these observations it was hypothesized that a single cell suspension of the CD13^{NEG}SSEA4^{POS}Tra-1-60^{POS} population could be generated by FACS to provide a highly enriched starting point for the formation of unique clones of iPS colonies. To this end CD13^{NEG}SSEA4^{POS} and CD13^{NEG}SSEA4^{POS}Tra-1-60^{POS} populations were sorted from adult skin fibroblasts at dpi8 directly into MEF coated 6 well plates and monitored the formation of colonies. (FIG. 1C) Small but distinct colony formation was observed in both the CD13^{NEG}SSEA4^{POS} and CD13^{NEG}SSEA4^{POS}Tra-1-60^{POS} sorted populations as early as 3 days post sorting (dps) with the sorted CD13^{NEG}SSEA4^{POS}Tra-1-60^{POS} populations producing more and larger colonies than the CD13^{NEG}SSEA4^{POS} populations. Following an additional two weeks of expansion without maintenance by manually removing differentiated cells, wells containing the CD13^{NEG}SSEA4^{POS} had become overgrown with differentiated cells but the sorted CD13^{NEG}SSEA4^{POS}Tra-1-60^{POS} wells contained large, well separated colonies with little differentiation occurring between them.

These data demonstrate that FACS can be used to enrich iPS cells which are viable, form colonies and contain very few transformed cells or adult fibroblasts.

To demonstrate the phenotypic and functional similarity between FACS and manually derived ips clones the fibroblast lines shown in FIG. 1B (0825, 1018, 1023) were reprogrammed in parallel wells under the retroviral protocol and standard derivation techniques or with FACS performed at dpi7.

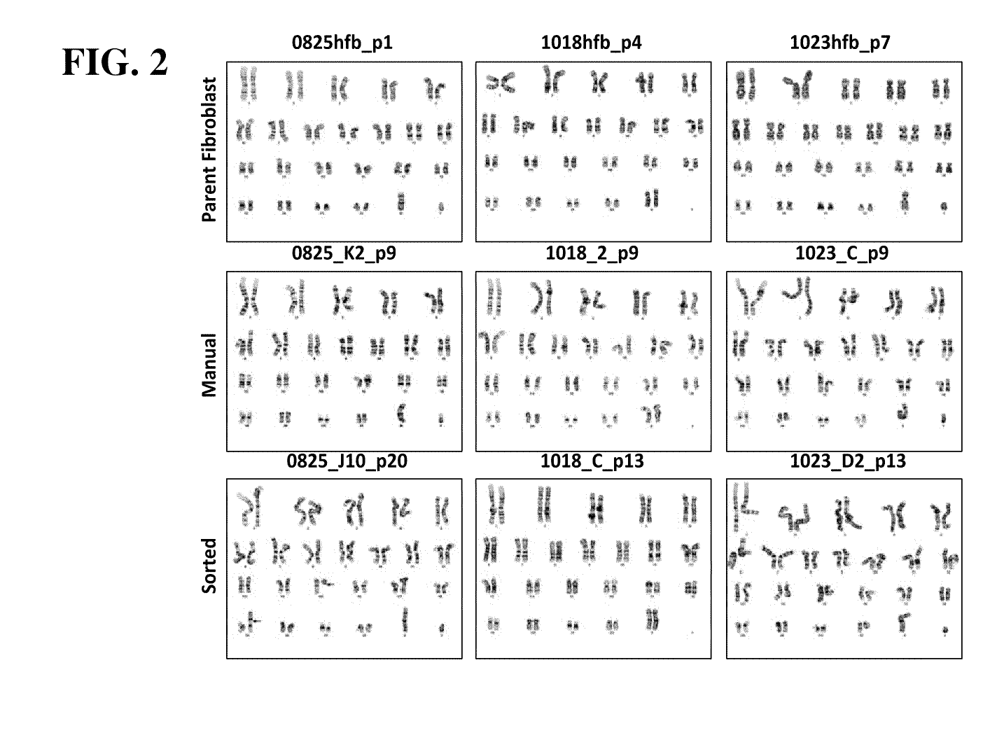

Several colonies possessing “good” qualities by eye were manually selected from each cell line and derivation technique and expanded prior to freezing. One clone from each line and technique was chosen for characterization. The karyotype of all derived clones matched the parent fibroblast line, displayed a normal karyotype and was free from of contamination with other cell lines. Table 5 and FIG. 2. Intriguingly, the parent 0825 fibroblast lines were found to be contaminated with an unknown cell lines most likely introduced into the tissue sample during time of tissue collection at the clinic. However, the cell lines derived by both techniques matched the parent fibroblast lines and were free of contamination with other cell lines.

TABLE 5
Karyotype of Parent Fibroblast and Reprogrammed Clones

| | 0825hfb_p5 | | | | | | |
|---|---|---|---|---|---|---|---|
| | Pre- | | | | Manual | | |
| | Dominant | CONTAMINATION | 1018hFB_p4 | 1023hFB_p7 | 0825_K2_p9 | 1018_2_p9 | 1023_C_p9 |
| Amelogenin | X, Y | X, Y | X | X, Y | X, Y | X | X, Y |
| vWA | 16 | 17, 19 | 16, 17 | 14, 18 | 16 | 16, 17 | 14, 18 |
| D8S1179 | 13, 15 | 12 | 10, 15 | 10, 14 | 13, 15 | 10, 15 | 10, 14 |
| TPOX | 9, 11 | 8 | 8 | 8, 11 | 9, 11 | 8 | 8, 11 |
| FGA | 20, 27 | 25 | 25, 27 | 20, 21 | 20, 27 | 25, 27 | 20, 21 |
| D3S1358 | 15, 16 | 17 | 15, 18 | 15, 18 | 15, 16 | 15, 18 | 15, 18 |
| TH01 | 7, 9 | — | 9.3 | 6, 8 | 7, 9 | 9.3 | 6, 8 |
| D21S11 | 27, 29 | 30, 31.2 | 28, 30 | 30, 30.2 | 27, 29 | 28, 30 | 30, 30.2 |
| D18S51 | 12, 19 | — | 13, 15 | 12, 13 | 12, 19 | 13, 15 | 12, 13 |
| Penta E | 8, 18 | 13, 16 | 12, 14 | 12, 16 | 8, 18 | 12, 14 | 12, 16 |
| D5S818 | 11, 12 | — | 11, 13 | 11 | 11, 12 | 11, 13 | 11 |
| D13S317 | 12, 14 | 9, 13 | 9, 11 | 10, 12 | 12, 14 | 9, 11 | 10, 12 |
| D7S820 | 8, 12 | 10 | 9, 10 | 8, 10 | 8, 12 | 9, 10 | 8, 10 |
| D16S539 | 10, 12 | 9, 11 | 11, 13 | 11, 12 | 10, 12 | 11, 13 | 11, 12 |
| CSF1PO | 7, 10 | 12 | 10, 12 | 10, 12 | 7, 10 | 10, 12 | 10, 12 |
| Penta D | 11, 13 | 9, 12 | 9, 13 | 12, 13 | 11, 13 | 9, 13 | 12, 13 |
| Karyotype | | Normal MALE | Normal | Normal | Normal | Normal | Normal |
| | | | FEMALE | MALE | MALE | FEMALE | MALE |
| Single Line | | | + | + | + | + | + |

| | Sorted | | |
|---|---|---|---|
| | 0825_J10_p20 | 1018_C_p13 | 1023_D2_p13 |
| Amelogenin | X, Y | X | X, Y |
| vWA | 16 | 16, 17 | 14, 18 |
| D8S1179 | 13, 15 | 10, 15 | 10, 14 |
| TPOX | 9, 11 | 8 | 8, 11 |
| FGA | 20, 27 | 25, 27 | 20, 21 |
| D3S1358 | 15, 16 | 15, 18 | 15, 18 |
| TH01 | 7, 9 | 9.3 | 6, 8 |
| D21S11 | 27, 29 | 28, 30 | 30, 30.2 |
| D18S51 | 12, 19 | 13, 15 | 12, 13 |
| Penta E | 8, 18 | 12, 14 | 12, 16 |
| D5S818 | 11, 12 | 11, 13 | 11 |
| D13S317 | 12, 14 | 9, 11 | 10, 12 |
| D7S820 | 8, 12 | 9, 10 | 8, 10 |
| D16S539 | 10, 12 | 11, 13 | 11, 12 |
| CSF1PO | 7, 10 | 10, 12 | 10, 12 |
| Penta D | 11, 13 | 9, 13 | 12, 13 |
| Karyotype | Normal | Normal | Normal |
| | MALE | FEMALE | MALE |
| Single Line | + | + | + |

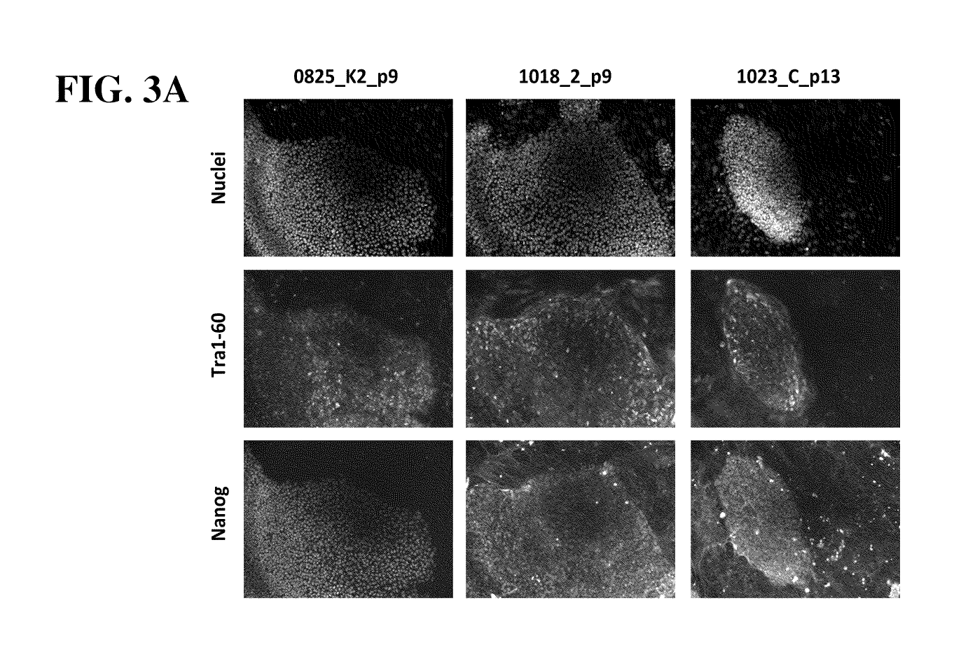

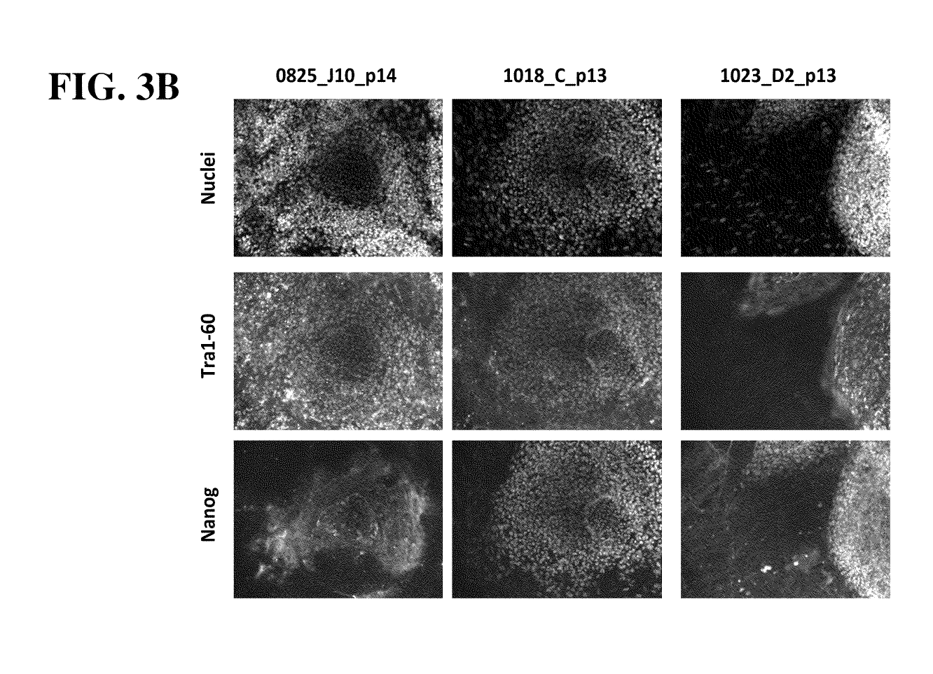

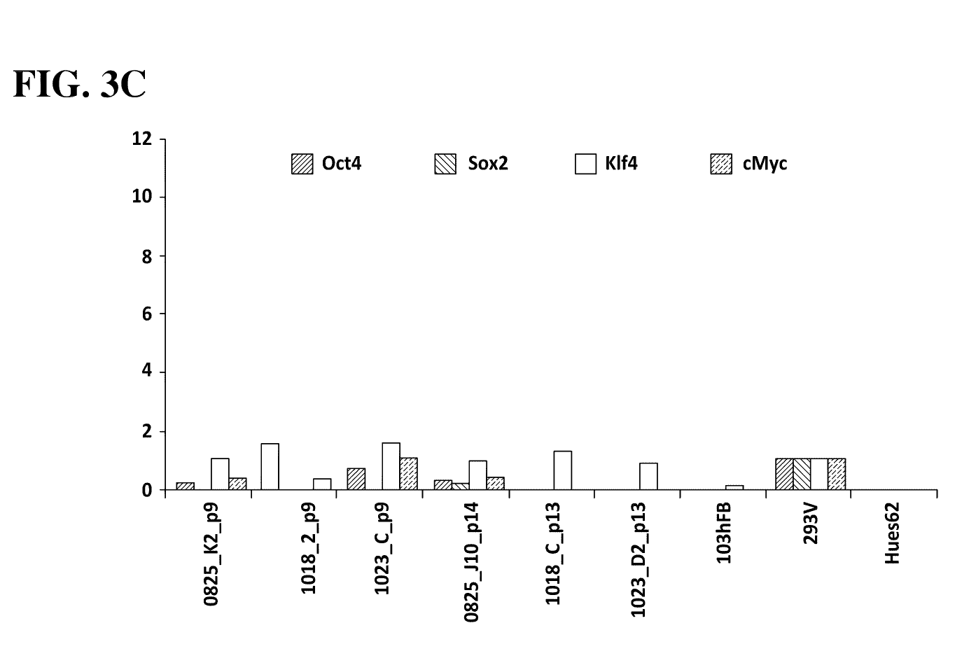

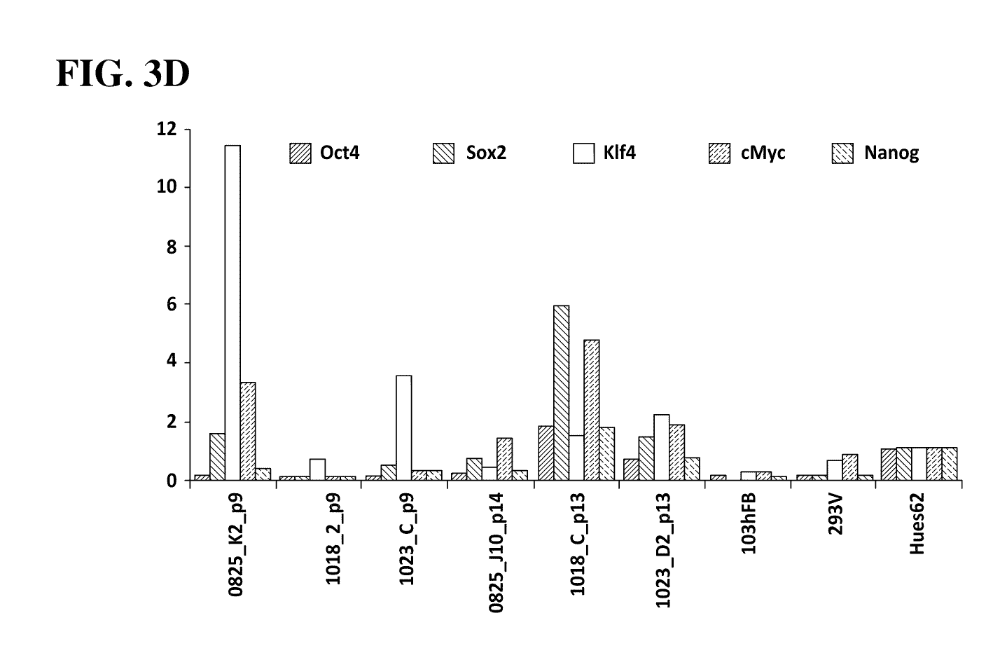

All iPSC lines generated compact colonies with morphology consistent with normal human embryonic stem cell (hESC) lines and were expanded for nine or greater passages prior to characterization. All lines expressed common markers of pluripotency by immunofluorescent microscopy, including the surface marker Tra-1-60 and the transcription factor Nanog (FIGS. 3A-B). Silencing of the virally transfected transcription factors Oct4, Sox2, Klf4, cMyc and the endogenous gene expression of Nanog, Oct4, Sox2, and Klf4 was confirmed by qPCR analysis (FIGS. 3C-D).

One advantage of using of FACS in derivation protocols is the capability of enriching rare reprogrammed cell populations from high passage fibroblast lines which show resistance to viral reprogramming techniques. A high passage fibroblast line was obtained from a commercial cell line repository, which failed to generate visually identifiable colonies after extended periods (>30 days) in culture.

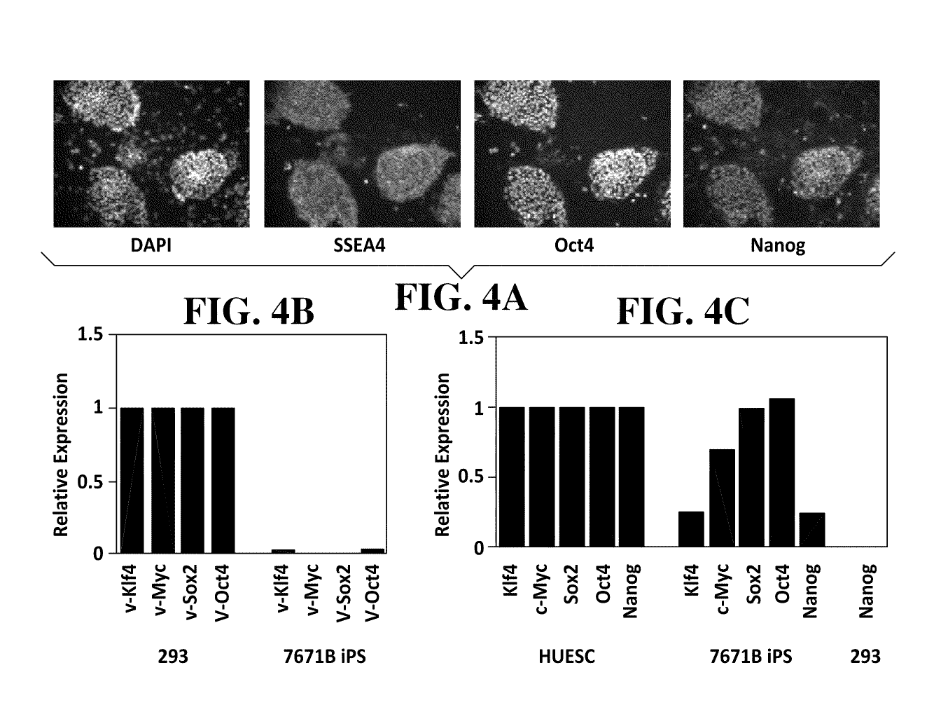

Samples of these cultures were dissociated and underwent FACS and produced colonies which were then manually picked, expanded and characterized. FACS derived iPS colonies displayed the pluripotent surface marker SSEA4 and transcription factors Oct4 and Nanog (FIG. 4A). Silencing of the virally transfected transcription factors Oct4, Sox2, Klf4, cMyc and the endogenous gene expression of Nanog, Oct4, Sox2, and Klf4 was confirmed by qPCR analysis (FIGS. 4B-4C).

To demonstrate the ability of FACS to derive clonally unique iPS lines generated by retroviral reprogramming protocols genomic DNA was harvested from manually, and FACS derived, cell lines and Southern blot analysis was performed to detect the presence of multiple DNA integration sites for the KLF4 and Oct4 transcription factors. Clones A and T derived from the 1023 lines appear to be the same clone as both contain the same integration sites for KLF4 (data not shown). The D2 clone derived by FACS displayed a unique integration pattern for KLF4 indicating a single clone. Additional lines derived by manual and FACS retroviral reprogramming protocols were tested and confirmed the existence of unique patterns of integration suggesting that FACS can be used to produce monoclonal cell lines under the retroviral protocol (data not shown).

Several groups have reported the existence of subpopulations lineage precursors that are identified by unique combinations of surface markers (Pruszak et al., 2009, Stem Cells 27: 2928-2940; Sundberg et al., 2009 Stem Cell Res 2: 113-124). It was hypothesized that there may be unique combinations of surface markers that may identify more fully reprogrammed cells within the CD13^{NEG}SSEA4^{POS}Tra-1-60^{POS} populations. A previous analysis using the 1018 ipS line suggested that the SSEA4^{POS}CD326^{POS} gated population expressed moderate to high levels of CD117, CD146, and CD49f and little to none CD49d, CD73, CD144, CD184, and CD309. This analysis was extended with the BD Lyoplate™ kit which consists of 242 purified antibodies to human Cluster of Differentiation (CD) surface markers. CD13^{NEG}SSEA4^{POS}Tra-1-60^{POS} populations from FACS or manually derived cell lines were sorted into 96 well plates and allowed to recover prior to incubation with the primary antibodies and image acquisition.

Consistent expression was observed in approximately 15% of surface markers contained in the Lyoplate™ kit, in at least 5 of the 9 lines tested, and variable expression of 4% of the surface markers in at least 3 of 9 lines tested. Based on these and the previous results of FC analysis it was determined to validate the expression of 48 surface markers on the SSEA4^{POS}Tra-1-60^{POS} populations by flow cytometry using the 0819 foreskin line. All cells withing the SSEA4^{POS}Tra-1-60^{POS} population expressed Alkaline Phosphatase, CD133, and CD326. To further define the reprogrammed population the characterization panel was adjusted to include CD236 and expression levels of 49 cell membrane surface markers on the SSEA4^{POS}Tra-1-60^{POS}CD326^{POS} population were evaluated.

Table 6 below indicates the 24 markers that were identified as being positively expressed on the SSEA4^{POS}Tra-1-60^{POS}CD326^{POS} population. The remaining 25 markers not expressed (negative) by the SSEA4^{POS}Tra-1-60^{POS}CD326^{POS} population are provided in Table 7, below.

TABLE 6
Positive Surface Marker Expression

| | Entrez | | |
|---|---|---|---|
| Marker | Number | Entrez Name | % Positive* |
| SSEA4 | — | Stage Specific Embryonic Antigen 4 | — |
| TRA-1-60 | 5420 | PODXL (podocalyxin-like) | — |
| CD326 | 17075 | Epcam epithelial cell adhesion molecule | — |
| SSEA3 | — | Stage Specific Embryonic Antigen 3 | 24 |
| TRA-1-81 | 5420 | PODXL (podocalyxin-like) | 70.5 |
| Alkaline | — | phosphate groups removing hydrolase | 100 |
| Phosphatase | | enzyme | |
| CD9 | 928 | transmembrane 4 superfamily, tetraspanin | 21.5 |
| | | family | |
| CD24 | 100133941 | Heat Stable Antigen | 99.7 |
| CD44 | 960 | Pgp-1, H-CAM, Ly24 | 14 |
| CD49c | 3675 | Integrin α3 chain, VLA-3 | 26 |
| CD49f | 3655 | Integrin α6 chain, VLA-6 | 98.2 |
| CD51/61 | 3685, 3690 | ITGAV (integrin, alpha V), ITGB3 | 0.8 |
| | | (integrin beta 3) | |
| CD57 | 27087 | B3GAT1 [beta-1,3-glucronyltransferase 1 | 99.4 |
| | | (glucuronosyltransferase P)] | |
| CD58 | 965 | LFA-3 | 45.3 |
| CD71 | 7037 | Transferrin Receptor | 11.3 |
| CD73 | 4907 | NT5E (5′-nucleotidase, ecto) | 0.5 |
| CD98 | 6520, 8140 | SLC3A2 [solute carrier family 3 | 99.5 |
| | | (activators of dibasic and neutral amino | |
| | | acid transport), member 2] | |
| CD117 | 3815 | KIT (v-kit Hardy-Zuckerman 4 feline | 9.6 |
| | | sarcoma viral oncogene homolog) | |
| CD133 | 8842 | PROM1 prominin 1 | 92 |
| CD146 | 4162 | MCAM (melanoma cell adhesion | 53.6 |
| | | molecule) | |
| CD193 | 1232 | (CCR3) chemokine (C-C motif) receptor 3 | 1232 |
| CD196 | 1235 | (CCR6) chemokine (C-C motif) receptor 6 | 17.9 |
| CD271 | 4804 | NGF Recepter | 2.3 |
| CD309 | 3791 | KDR (kinase insert domain receptor (a | 0.97 |
| | | type III receptor tyrosine kinase)) | |
| CD338 | 9429 | CDw338 (ABCG2) | 0.88 |

*Indicates proportion of SSEA4^{POS}Tra-1-60^{POS}CD326^{POS} expressing the specified marker

Negative Surface Marker Expression

| | Entrez | |
|---|---|---|
| Marker | Number | Entrez Name |
| CD13 | 290 | ANPEP alanyl (membrane) aminopeptidase) |
| D7Fib | — | |
| CD1b | 910 | |
| CD3 | 916 | |
| CD34 | 947 | gp 105-120 |
| CD40 | 958 | TNF receptor superfamily member 5 |
| CD45RA | 5788 | PTPRC (protein tyrosine phosphatase, receptor |
| | | type, C) |
| CD49a | 3672 | Integrin α1 chain |
| CD49b | 3673 | Integrin α2 chain, VLA-2 |
| CD49d | 3676 | Integrin α4 chain, VLA-4 |
| CD99 | 4267 | |
| CD106 | 7412 | VCAM1 |
| CD107a | 3916 | LAMP1 |
| CD107b | 3920 | LAMP2 |
| CD116 | 1438 | CSF2RA [colony stimulating factor 2 receptor, |
| | | alpha, low-affinity] (GM-CSF Receptor) |
| CD137L | 8744 | TNFSF9 (tumor necrosis factor (ligand) |
| | | superfamily, member 9) (4-1BBLigand) |
| CD144 | 1003 | CDH5 [cadherin 5, type 2 (vascular |
| | | endothelium)] |
| CD147 | 682 | BSG [basigin (Ok blood group)] (Neurothelin) |
| CD164 | 8763 | |
| CD166 | 214 | ALCAM (activated leukocyte cell adhesion |
| | | molecule) |
| CD184 | 7852 | (CXCR4, Fusin) |
| CD195 | 1234 | (CCR5) |
| CD235a | 2993 | GYPA (Glycophorin A) |
| CD243 | 5243 | ABCB1 [ATP-binding cassette, sub-family B |
| | | (MDR/TAP), member 1] [P-glycoprotein (MDR)] |

### Comparision of Retrovirus and Sendai Virus Vectors in Reprogramming

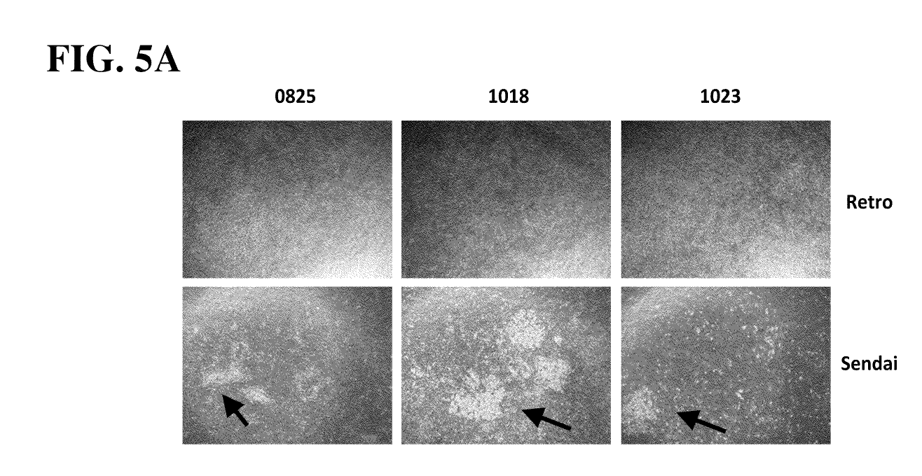

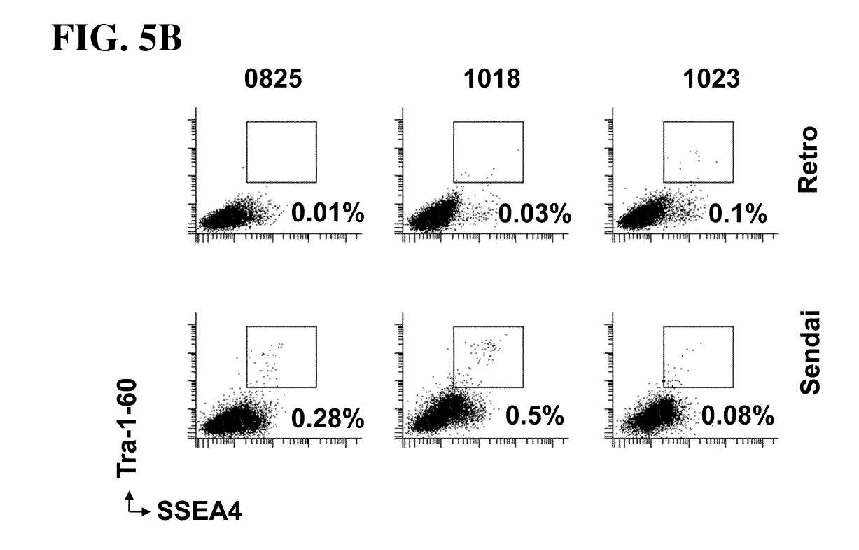

To further increase reprogramming efficiency and avoid the issue of polyclonalism due to multiple DNA integration the Sendai virus was tested as a reprogramming vector relative to the retroviral construct. At dpi7 colony formation was observed in the Sendai virus infected well and no colonies in the retroviral infected wells. (FIG. 5A) Wells imaged in FIG. 5A underwent analysis by FC to detect expression levels of the CD13^{NEG}SSEA4^{POS}Tra-1-60^{POS} populations. (FIG. 5B) Very few CD13^{NEG}SSEA4^{POS}Tra-1-60^{POS} populations were present in the retrovirally reprogrammed fibroblasts but were present in higher proportions in the fibroblasts reprogrammed using the Sendai virus. The 1023 line was more efficiently reprogrammed using the retroviral technique suggesting that certain cell lines may be more efficiently reprogrammed by one method over another. Three additional cell lines were FACS derived at dpi7 under the Sendai protocol and showed a similar trend of early visible colony formation at dpi7 and high proportions of the CD13^{NEG}SSEA4^{POS}Tra-1-60^{POS} populations.

### INCORPORATION BY REFERENCE

Numerous references are cited hereinabove, all of which are incorporated herein by reference in their entireties.

We claim:

1. A method for producing induced pluripotent stem cells (iPSC) from adult fibroblasts comprising the steps of:
(a) contacting adult fibroblasts with a reprogramming composition suitable for reprogramming the adult fibroblasts to iPSC under conditions effective for the reprogramming composition to penetrate the adult fibroblasts,
(b) culturing the contacted fibroblasts for a time period sufficient for the cells to be reprogrammed;
(c) sorting the cultured cells to select cells based upon their expression of the cell membrane surface markers CD13^{NEG} SSEA4^{POS} Tra-1-60^{POS}.
(d) identifying iPSC colonies from the sorted cells of (c).

2. The method of claim 1 wherein the reprogramming composition comprises at least one expression vector expressing a transcription factor suitable for reprogramming the adult fibroblasts to iPSC.

3. The method of claim 2 wherein the expression vector expresses at least one transcription factor that is selected from the group consisting of Oct4, KLF4, Sox2, Lin28, Nanog, c-Myc, 1-Myc and combinations thereof.

4. The method of claim 3 wherein the transcription factor is selected from the group consisting of Oct4, KLF4, Sox, c-Myc and combinations thereof.

5. The method of claim 1 where the reprogramming composition further comprises inhibitors of pathways selected from the group consisting of TGFb pathway, MAPK/ERK pathway, GSK3 pathway, histone deacetylase (HDAC) inhibitors, activators of 3′-phosphoinositide-dependent kinase-1 (PDK1), mitochondrial oxidation modulators, glycolytic metabolism modulators, HIF pathway activators and combinations thereof.

6. The method of claim 5 wherein the pathway inhibitors are selected from the group consisting of SB431542, A-83-0, PD0325901, CHIR990, Parnate, PS48, sodium butyrate, valproic acid, 2,4-dinitrophenol, fructose 2,6-bisphosphate, oxalate, N-oxaloylglycine, Quercetin and combinations thereof.

7. The method of claim 1 wherein the identifying step (d) comprises visually screening and identifying those clones having the appearance of iPSC colonies.

8. The method of claim 1 wherein the identifying step (d) comprises testing the sorted cells by flow cytometry (FC) or immunofluorescent (IF) microscopy to identify those cells with positive expression levels for a cell membrane surface markers selected from the group consisting of alkaline phosphatase, SSEA3, Tra-1-81, CD326 and combinations thereof.

9. The method of claim. 1 wherein the identifying step (d) comprises testing the sorted cells by flow cytometry (FC) or immunofluorescent (IF) microscopy to identify those cells with positive expression levels for cell membrane surface markers selected from the group consisting of CD9, CD24, CD44, CD49c, CD49f, CD51161, CD57, CD58, CD71, CD73, CD98, CD117, CD133, CD146, CD193, CD196, CD271, CD309, CD33 8 and combinations thereof.

10. The method of claim 1 wherein the identifying step (d) comprises testing the sorted cells by flow cytometry (FC) or immunofluorescent (IF) microscopy to identify those cells with positive expression levels for nuclear located transcription factors Oct 4, KLF4, Sox2, Nanog and combinations thereof.

11. The method of claim 1 wherein the identifying step (d) comprises testing the sorted cells by Southern blotting of the sorted cells to identify unique clones based on patterns of viral DNA integration of Oct 4, KLF4, Sox2, or c-Myc transcription factors in the sorted cells.

12. The method of claim 1 wherein the identifying step (d) comprises testing by quantitative real time PCR of the sorted cells to detect silencing of the retrovirally induced transcription factors Oct 4, KLF4, Sox2, or c-Myc and the endogenous expression of the Oct 4, KLF4, Sox2, or Nanog transcription factors in the sorted cells.

13. The method of claim 1 wherein the identifying step (d) comprises testing by teratoma formation by the sorted cells in immunocompromised mice to confirm the ability of the sorted cells to form all three germ layers.

14. The method of claim 1 wherein the identifying step (d) comprises testing by inducing the sorted cells to form Embryoid Bodies, thereby confirming the ability of the sorted cells to form all three germ layers.

15. The method of claim 1 wherein the identifying step (d) comprises testing by FACS enriching the CD13^{NEG} SSEA4^{POS} Tra-1-60^{POS} sorted cell populations into multiwell plates for high throughput derivation assays.

16. The method of claim 1 wherein the adult fibroblasts are high passage fibroblasts.

17. The method of claim 1 wherein the adult fibroblasts are obtained from biopsy tissue or are fibroblasts contaminated with known or unknown cell lines.

18. The method of claim 1 wherein the fibroblasts are human fibroblasts.

19. The method of claim 1 wherein the expression vector is selected from the group Consisting of a retrovirus, a lentivirus, an adenovirus, an adeno associated virus, a herpes virus, a Sindbis virus, a pox virus, a bacula virus, a bacterial phage, a Sendai virus and combinations thereof.

20. The method of claim 19 wherein the Sendai virus is a nonreplicative virus.

21. The method of claim 1 wherein step (a) is conducted by electroporation, chemical transfection or by means of cell penetrating proteins, of the adult fibroblasts.

22. The method of claim 1 wherein the reprogramming composition comprises an RNA, a protein or a small molecule.

23. The method of claim 22 wherein the RNA is selected from the group consisting of mRNA, microRNA, siRNA, antisense RNA and combinations thereof.

24. The method of claim 21 wherein the chemical transfection is conducted by means of a chemical transfecting agent selected from the group consisting of a cationic lipid, a polymer, calcium phosphate and combinations thereof.

25. The method of claim 21 wherein the cell penetrating protein is selected from the group consisting of a TAT tagged protein and an arginine rich protein.

26. The method of claim 25 wherein the arginine rich protein is selected from the group consisting of protein.

27. The method of claim 5 wherein the mitochondrial oxidation modulator is 2,4-dinitrophenol.

28. A composition comprising pluripotent stem cells produced by the method of claim 1.


SEQUENCE LISTING (24 sequences)

SEQ ID NO: 1 - DNA, 23 bp, homo sapiens
ccccagggccccattttggtacc

SEQ ID NO: 2 - DNA, 20 bp, homo sapiens
ggcacaaactccaggttttc

SEQ ID NO: 3 - DNA, 20 bp, homo sapiens
acactgcccctctcacacat

SEQ ID NO: 4 - DNA, 22 bp, homo sapiens
gggttttctccatgctgtttct

SEQ ID NO: 5 - DNA, 22 bp, homo sapiens
acccacacaggtgagaaacctt

SEQ ID NO: 6 - DNA, 22 bp, homo sapiens
gttgggaacttgaccatgattg

SEQ ID NO: 7 - DNA, 22 bp, homo sapiens
agcagaggagcaaaagctcatt

SEQ ID NO: 8 - DNA, 22 bp, homo sapiens
ccaaagtccaatttgaggcagt

SEQ ID NO: 9 - DNA, 23 bp, homo sapiens
ccccagggccccattttggtacc

SEQ ID NO: 10 - DNA, 22 bp, homo sapiens
aacctacaggtggggtctttca

SEQ ID NO: 11 - DNA, 20 bp, homo sapiens
acactgcccctctcacacat

SEQ ID NO: 12 - DNA, 22 bp, homo sapiens
aacctacaggtggggtctttca

SEQ ID NO: 13 - DNA, 20 bp, homo sapiens
gaccacctcgccttacacat

SEQ ID NO: 14 - DNA, 22 bp, homo sapiens
aacctacaggtggggtctttca

SEQ ID NO: 15 - DNA, 22 bp, homo sapiens
agcagaggagcaaaagctcatt

SEQ ID NO: 16 - DNA, 22 bp, homo sapiens
aacctacaggtggggtctttca

SEQ ID NO: 17 - DNA, 19 bp, homo sapiens
tagctgtgctcgggctact

SEQ ID NO: 18 - DNA, 18 bp, homo sapiens
tctctgctggatgacgcg

SEQ ID NO: 19 - DNA, 20 bp, homo sapiens
gagaaggagaagctggagca

SEQ ID NO: 20 - DNA, 20 bp, homo sapiens
gtgaagtgagggctcccata

SEQ ID NO: 21 - DNA, 20 bp, homo sapiens
agaaccccaagatgcacaac

SEQ ID NO: 22 - DNA, 20 bp, homo sapiens
tggagtgggaggaagaggta

SEQ ID NO: 23 - DNA, 20 bp, homo sapiens
acctggcgagtctgacatgg

SEQ ID NO: 24 - DNA, 23 bp, homo sapiens
tcttcatgtgtaaggcgaggtgg